(19) United States
(12) Patent Application Publication
(10) Pub. No.: US 2019/0043623 A1
(43) Pub. Date: Feb. 7, 2019

(54) SYSTEM AND METHOD FOR PHYSIOLOGICAL AND PSYCHOLOGICAL SUPPORT IN HOME HEALTHCARE

(71) Applicant: Thomas W. WATLINGTON, IV, La Mesa, CA (US)

(72) Inventor: Thomas W. WATLINGTON, IV, La Mesa, CA (US)

(21) Appl. No.: 16/055,060

(22) Filed: Aug. 4, 2018

Related U.S. Application Data
(60) Provisional application No. 62/541,443, filed on Aug. 4, 2017.

(51) Int. Cl. G16H 80/00; G16H 20/70; G10L 15/26; G10L 13/04; G08B 21/02

(52) U.S. Cl. CPC G16H 80/00; G16H 20/70; G08B 21/02; G10L 13/043; G10L 15/265

(57) ABSTRACT

A voice-enabled ambient personal assistant positioned in a place of residence of an individual, or carried on the person of the individual is provided to help the individual feel safe, remain close to friends, family, and community, and to instill a feeling that life continues to be meaningful in order to live independently in their home safely for as long as possible. Moderator(s) are able to control the information being communicated through the personal assistant to the individual or group of individuals to personalize the information to the memories, tastes, and/or needs of the individual.

The present application claims the benefit of Unites States Provisional Patent Application 62/541,443, filed Aug. 4, 2017, which is hereby incorporated by reference in its entirety including all tables, figures, and claims.

### BACKGROUND OF THE INVENTION

The following description is provided simply as an aid in understanding the invention and is not admitted to describe or constitute prior art.

According to the 2010 Census, there were 40.3 million people 65 and older on Apr. 1, 2010, increasing by 5.3 million since the 2000 Census when this population numbered 35.0 million. The percentage of the population 65 and older also increased during the previous decade. In 2010, the older population represented 13.0 percent of the total population, an increase from 12.4 percent in 2000. This population is particularly prone to chronic conditions including Alzheimer's disease, hypertension, heart disease, mental illness, diabetes, arthritis, cancer, back problems, and COPD, and it is estimated that over 60% of those age 65 and older have two or more chronic conditions. How to care for these elderly and disabled effectively is critical to preventing repeated hospital admissions that result in escalating healthcare costs.

As most people age, suffer from chronic diseases, or become handicapped physically or mentally, they generally prefer to live at home for as long as possible rather than move into a long term care institution or hospital. One option to extend a person's time at home before moving into a long term care institution is home care. As a general rule, with the proper care, most people can live out their lives at home without ever entering a care facility. In-home care services can be very expensive, however, especially in cases where continuous supervision is required.

In the United States, Medicare will cover a limited amount of home care expenses for some patients having certain non-permanent conditions, but Medicare will not cover long term home care expenses, nor will it reimburse for medical devices. When Medicare benefits have expired and there is little or no money available for a person's care, a decision is often made to use Medicaid. Medicaid will pay for certain aspects of home care, but only while it is more cost effective and more efficient than a nursing home, which is rarely the case. Thus, only those families with substantial assets or income are able to continue home care with professional services. Consequently, a lack of financial resources typically is the primary reason why most persons are unable to take advantage of in-home care services.

### BRIEF DESCRIPTION OF THE FIGURES

FIG. 1 depicts an exemplary architecture for a personal assistant/reporting system of the invention.

FIG. 2 depicts an exemplary architecture for a recommendation engine receiving and processing event information for an individual.

FIG. 3 depicts an exemplary architecture for a recommendation engine receiving and processing question information for an individual.

### DETAILED DESCRIPTION OF THE INVENTION

The present invention relates to methods and systems for physiological and psychological support of individuals that require in-home care services. In various aspects, the present invention makes use of a voice-enabled ambient personal assistant positioned in a place of residence of an individual, or carried on the person of the individual (for example as a smartphone or tablet) to help the individual feel safe, remain close to friends, family, and community, and to instill a feeling that life continues to be meaningful in order to live independently in their home safely for as long as possible.

The personal assistant is under control of one or more moderators (for example, a family member, or care-giver such as a nurse, physician or close friend), and may be accessed by the moderator(s) from a remote location to control the information being communicated through the personal assistant to the individual or group of individuals. In this way, the moderator may personalize the information to the memories, tastes, and/or needs of the individual. For example, the information may include music, photos, voices, etc., that are meaningful to the individual(s). The information may also include reminders and/or questions concerning actions that need to be taken on a particular day (medications, nutrition, appointments, arrival of in-home aides, etc.).

The information may also include assessments which require the individual(s) to respond, and from which decisions can be made concerning the status of the individual. For example, screening and monitoring tools such as the online assessment measures described in Section III of DSM-5; the Patient Stress Questionnaire adapted from adapted from PhQ 9, GAD7, PC-PTSD and AUDIT 1/24/11; the Columbia Suicide Severity Rating Schedule, etc., may be administered and used to assess the mental health and cognitive ability of the individual. Other assessments may include prompting for physiological measures such as blood pressure, weight, oxygen saturation, etc., which may be easily assessed in the home by the individual.

A database, preferably provided as a cloud-based computing service, may be accessed by the moderator(s) in order to receive and store instructions for controlling communication through the personal assistant. The database may be accessed via a web-based software or smartphone app which permits the moderator(s) to customize the interaction between the individual and the personal assistant. This flexibility permits the moderator(s) to maintain customization to the individual as circumstances change over time and new issues can arise with regard to physical, emotional or sensory abilities. The data transmitted to the individual through the personal assistant may be regulated in this manner (e.g., as to content, timing, frequency, etc.) by the moderator(s).

Because the personal assistant ambiently samples sounds in the place of residence, the individual need not learn any new technology in order to receive the benefits of the system; rather, the individual may simply speak to the personal assistant which, because it is voice enabled, will speak to the individual. The personal assistant preferably transfers the sounds received to the cloud-based service, which collects and processes the available sounds using natural language processing software to convert the sounds to language, and the language may be interpreted to understand the needs of the individual and responses to the questions related to the physiological and psychological health of the individual. The processed language can then be stored in the database record of the individual. Communication between the individual and the personal assistant may be initiated by either the individual, or by the cloud-based service through the personal assistant.

In addition, the information communicated by the personal assistant may take the form of a chatbot, or other agent designed to have conversations with a human. An example of such a chatbot is described in U.S. Pat. No. 9,369,410. An initial set of profile variables may be defined by the moderator(s), and can be combined with the content of conversations in order to tailor the content of the conversation to relevant characteristics such as the individual's interests, personality and demographic. The profile variables that are used to build the chatbot, and the processed language resulting from interaction with the chatbot, may be stored in the database record of the individual, and modified under the control of the moderator(s).

In certain embodiments, the information communicated by the personal assistant may be generated, guided, or regulated by a machine learning model, trained on the prior interactions of individuals and moderators with the system, as well as taking into account profile variables and other features in the database. Online training may be utilized for continuous model updates, to facilitate real-time responsiveness and dynamic interactions for the individuals and moderators. An example of such a machine learning model is described in WO2018098060A1 and US20170230312A1.

In certain embodiments, a need for urgent response can be noted by the software based on the processed sounds, and this need can be communicated immediately to the moderator(s) and to emergency responders.

In certain embodiments, the processed language stored in the database for the individual may be processed in order to generate a report for the individual. This report may be viewed by the moderator(s), and/or delivered to one or more recipients designated by the moderator(s). The report may analyze the processed language for changes in mood, need to involve health care provider(s), changes in habits, changes in cognition, etc. In addition, names of relatives and friends that are brought up by the individual (e.g., “I have not heard from Jack or Jill for a while”) can be used to trigger a visit or call from the person involved or content of photos, music, sounds, etc. may be triggered that are meaningful based on the particular relatives and friends involved. Subjects such as shopping, visiting a favorite community spot, need for replenishment of household staples, etc., can be noted in the report, and activities relevant to the subject can be scheduled.

In certain embodiments, the report comprises a literal transcription of the sounds received by the personal assistant and that is processed using the natural language processing software into language. The literal transcript can further provide a correlation of specific sentences received by the personal assistant to a date and time of their receipt.

The report may also comprise one or more numerical parameters calculated to correspond to the physiological and/or psychological health of the individual that is based on the sounds received by the personal assistant (e.g., question responses, chatbot conversations, or spontaneous comments from the individual). An alert may be transmitted to the moderator and/or one or more recipients designated by the moderator if one or more of the parameters deviates from a predetermined rule in a manner that indicates a potential issue with the individual.

The same method of calculating one or more numerical parameters selected to correspond to the physiological and/or psychological health of an individual can be applied for a group of individuals controlled and reviewed by the same moderator(s).

In another embodiment, numerical parameters calculated to correspond to the physiological and/or psychological health of the individual and based on the sounds received by the personal assistant (e.g., question responses, chatbot conversations, or spontaneous comments from the individual) can also be delivered as a report to the individual(s) in either a verbal or written form, in response to a question about their own performance in an area being tracked such as medication, exercise, or other health related or as a composite response to the general question, “how am I doing taking care of myself?” The answer can be quantitative as well as provide advice to the individual regarding behavioral changes to improve the score.

The software can be personalized to reflect the goals and parameters of interest by the individual or the moderator(s). The process of personalization then defines the frequency and nature of questions asked by the voice-assistant software related to matters of health so that the proper emphasis is given to metrics that reflect the individuals own health interests. For example the individual or their moderator may not be interested in tracking exercise but nutrition and medication compliance being of great interest are thus weighted in the algorithm that determines the questions that are asked of the individual by the software.

In certain embodiments, the personal assistant may also be operably connected to one or more additional sensors within the residence which are used to monitor habits of the individual or to record movement, analyze facial and other indicia of mood and emotional state. For example, a sensor on the refrigerator or kitchen storage area can be used to infer that the individual is eating, and changes in the time or regularity of triggering of such sensors may indicate a change in habits that is noted on the report. With regard to emotional state, recording of facial expressions and posture may be analyzed for interest in content being viewed, emotions, depression, anger, etc. See, e.g., US20140315168; US9202110. Correlating the data from the physical sensors with the data generated by the individual interacting with the voice-assisted software provides a form positive or negative validation which can be added to the report.

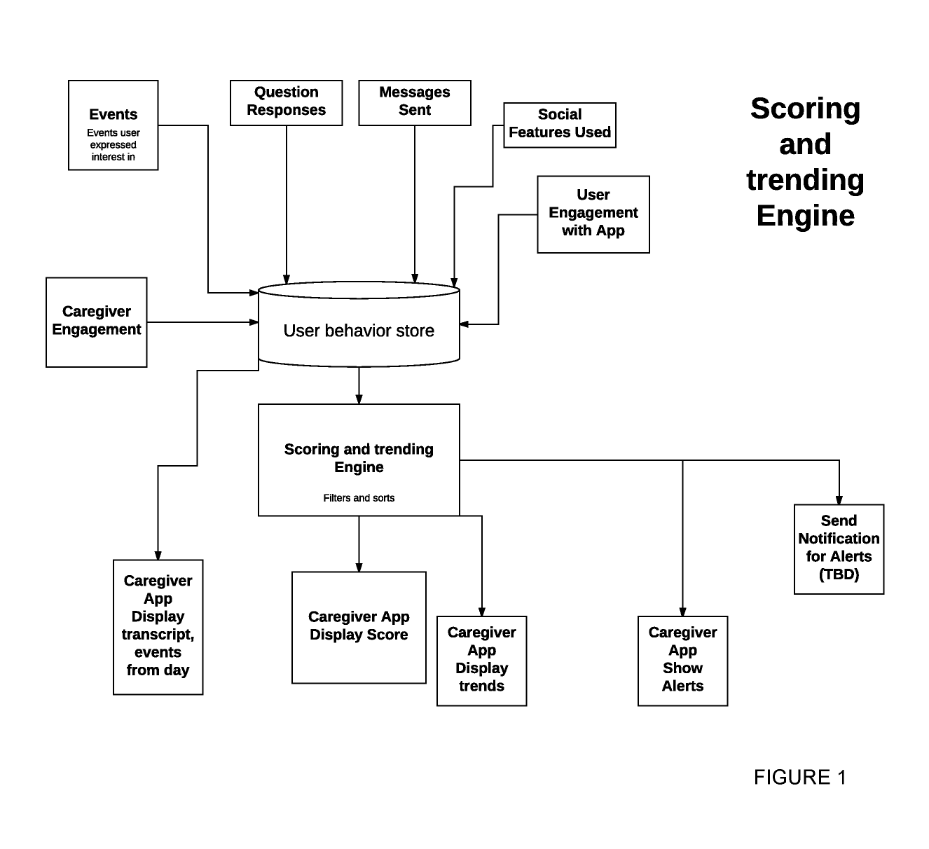

FIG. 1 depicts an exemplary architecture for a personal assistant/reporting system of the invention. As depicted, inputs can include interests or events that are meaningful to the individual, responses to questions, a list or summary of messages sent by the individual, any features used by the individual recently of a social interaction nature, a level or mood of engagement with the personal assistant, and a level of caregiver engagement with the personal assistant (e.g., number of times used per day, duration of usage, messages sent and received). These can be scored by a scoring engine for negative or positive emotional state, goals, desires, health, etc. These can also be stored within the individual's “behavior store” and used to analyze trends in these values by a trending engine.

The scoring and trends engine uses user behavior data to produce scores for mood and wellness, exercise and medication compliance, and social engagement. It also analyzes user behavior for trends, and produces alerts to notify the caregiver according to their notification preferences. Outputs can be as simple as a raw transcript of the inputs, or can be made more granular by reporting calculated scores and trends. If the score or trend is indicative of a potential health issue or that a particular response by a moderator would be advantageous, an alert or alarm may be presented.

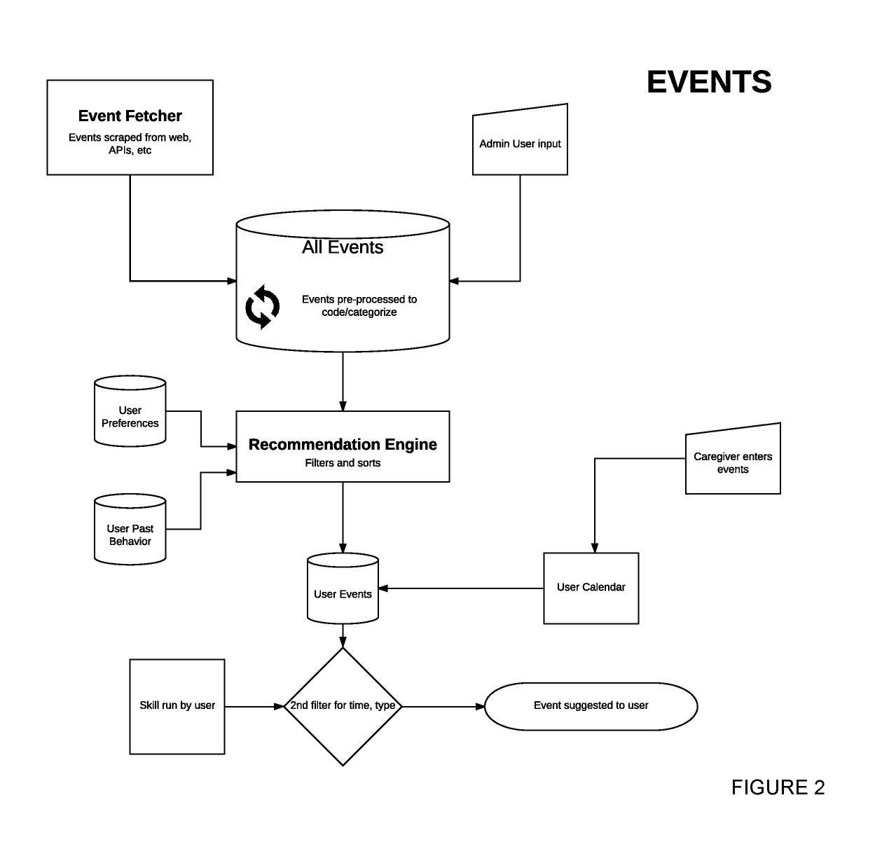

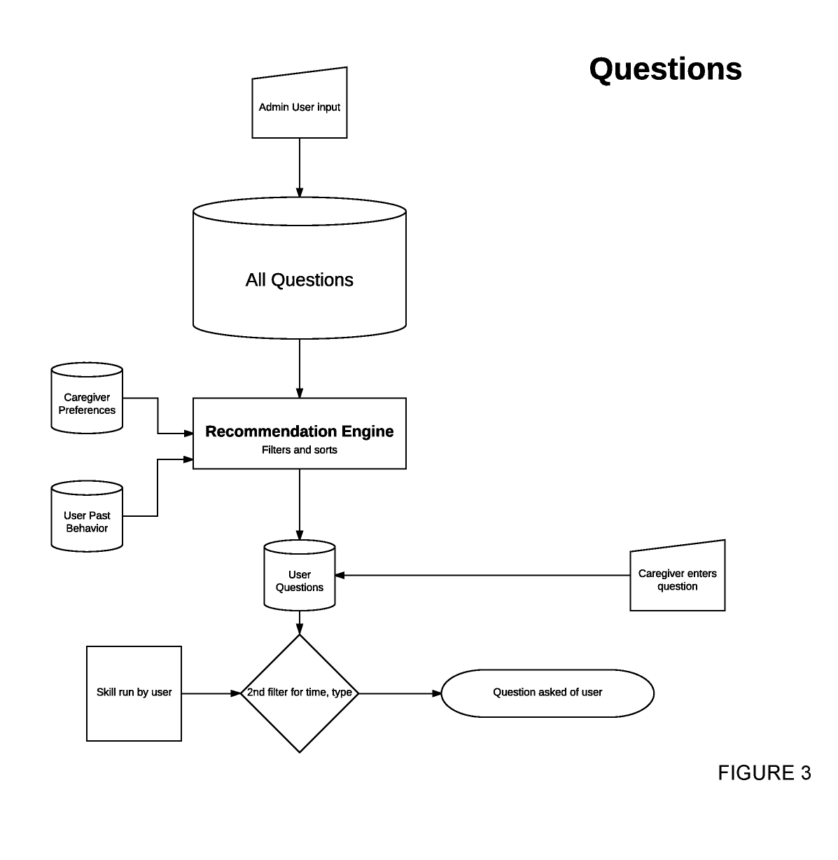

A recommendation engine filters and sorts events, questions and reminders presented to the user as depicted in FIGS. 2 and 3. The engine rankings can be based on static rules as well as dynamic learning, such as collaborative filtering and predictive models. Recommendations determined to have high priority or time-sensitivity can be proactively pushed, without requiring a user to initiate engagement. The recommendation engine can expose users to novel events and encourage behaviors that may be associated with an individual's trending and scoring, thus forming a positive feedback cycle towards improving outcomes. By way of example, the recommendation engine may use the following inputs:

Location of the individual

### Time of day

Capabilities of the individual
Interests of the individual
Last answer by user for this question/event category (e.g. if they answered “No” to are you well/did you sleep well, ask it again soon to see if pattern)
Trends of user behavior (e.g. reduce frequency of med reminder question if they are always compliant)
Question/Event related:
Frequency of this question (e.g. if asked yesterday, wait to ask again)
Relation of this event to other recent suggestions to be presented (e.g. don't present 3 exercises in a row that are somewhat similar).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed.

Thus, it should be understood that although the present invention has been specifically disclosed by preferred embodiments and optional features, modification, improvement, and variation of the inventions disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. Other embodiments are set forth within the following claims.

What is claimed is:

1. A method of collecting and storing information related to the physiological and psychological health of an individual under the control of a moderator, comprising:
providing the moderator with an application interface that provides the moderator with remote control of a voice-enabled ambient personal assistant (APA) positioned in a place of residence of the individual or carried on their person, wherein the application interface is operably connected to a cloud-based computing service comprising a database which stores outgoing data that is communicated to the individual through the APA in a database record that is specific to the individual, wherein the outgoing data is regulated by the moderator, and wherein the outgoing data comprises questions related to the physiological and psychological health of the individual;
communicating the outgoing data to the individual using the APA, wherein the APA is operably connected to the cloud-based computing service, and wherein the cloud-based service collects vocal sounds made at the place of residence, processes the vocal sounds using natural language processing software to determine responses to the questions related to the physiological and psychological health of the individual, and stores the responses in the database record that is specific to the individual;
summarizing the responses in a report generated by the cloud-based computing service and distributing the report to one or more recipients designated by the moderator.

2. A method according to claim 1, wherein the outgoing data is regulated by the moderator according to frequency of communication and/or content of data.

3. A method according to claim 1, wherein the outgoing data comprises statements or questions from a software agent designed to have conversations with a human.

4. A method according to claim 2, wherein the outgoing data comprises statements or questions from a software agent designed to have conversations with a human.

5. A method according to claim 1, wherein the report comprises a literal transcription of language processed from the vocal sounds processed using the natural language processing software, which are optionally correlated to a date and time of receipt of the vocal sounds by the APA.

6. A method according to claim 2, wherein the report comprises a literal transcription of language processed from the vocal sounds processed using the natural language processing software, which are optionally correlated to a date and time of receipt of the vocal sounds by the APA.

7. A method according to claim 3, wherein the report comprises a literal transcription of language processed from the vocal sounds processed using the natural language processing software, which are optionally correlated to a date and time of receipt of the vocal sounds by the APA.

8. A method according to claim 4, wherein the report comprises a literal transcription of language processed from the vocal sounds processed using the natural language processing software, which are optionally correlated to a date and time of receipt of the vocal sounds by the APA.

9. A method according to claim 1, wherein the report comprises one or more numerical parameters calculated to correspond to the physiological and/or psychological health of the individual.

10. A method according to claim 2, wherein the report comprises one or more numerical parameters calculated to correspond to the physiological and/or psychological health of the individual.

11. A method according to claim 3, wherein the report comprises one or more numerical parameters calculated to correspond to the physiological and/or psychological health of the individual.

12. A method according to claim 4, wherein the report comprises one or more numerical parameters calculated to correspond to the physiological and/or psychological health of the individual.

13. A method according to claim 5, wherein the report comprises one or more numerical parameters calculated to correspond to the physiological and/or psychological health of the individual.

14. A method according to claim 6, wherein the report comprises one or more numerical parameters calculated to correspond to the physiological and/or psychological health of the individual.

15. A method according to claim 7, wherein the report comprises one or more numerical parameters calculated to correspond to the physiological and/or psychological health of the individual.

16. A method according to claim 8, wherein the report comprises one or more numerical parameters calculated to correspond to the physiological and/or psychological health of the individual.

17. A method according to claim 9, wherein an alert is transmitted to the moderator and/or one or more recipients designated by the moderator if one or more of the parameters deviates from a predetermined rule.

18. A method according to claim 10, wherein an alert is transmitted to the moderator and/or one or more recipients designated by the moderator if one or more of the parameters deviates from a predetermined rule.

19. A method according to claim 11, wherein an alert is transmitted to the moderator and/or one or more recipients designated by the moderator if one or more of the parameters deviates from a predetermined rule.

20. A method according to claim 12, wherein an alert is transmitted to the moderator and/or one or more recipients designated by the moderator if one or more of the parameters deviates from a predetermined rule.

21. A method according to claim 13, wherein an alert is transmitted to the moderator and/or one or more recipients designated by the moderator if one or more of the parameters deviates from a predetermined rule.

22. A method according to claim 14, wherein an alert is transmitted to the moderator and/or one or more recipients designated by the moderator if one or more of the parameters deviates from a predetermined rule.

23. A method according to claim 15, wherein an alert is transmitted to the moderator and/or one or more recipients designated by the moderator if one or more of the parameters deviates from a predetermined rule.

24. A method according to claim 16, wherein an alert is transmitted to the moderator and/or one or more recipients designated by the moderator if one or more of the parameters deviates from a predetermined rule.
